(19) United States
(12) Patent Application Publication
(10) Pub. No.: US 2006/0178717 A1
(43) Pub. Date: Aug. 10, 2006

(54) Adjustable disposable surgical thermal blanket

(76) Inventors: Christa Harris, Waukegan, IL (US); Agnieszka Przeklasa, Mundelein, IL (US); Eric Ellingson, Libertyville, IL (US)

Correspondence Address: Kimberly Luna, Paralegal;Allegiance Corporation, 1430 Waukegan Road, McGaw Park, IL, 60085, US

(21) Appl. No.: 11/055,323

(22) Filed: Feb. 10, 2005

(51) Int. Cl. A61F 7/00

(52) U.S. Cl. 607/114

(57) ABSTRACT

The invention described herein is a disposable, adjustable patient warming blanket comprising: a) a first blanket sheet and a second blanket sheet, said sheets being joined to one another around the perimeter to form a generally planar containment structure having a first side and second side and an exterior surface and interior surface, at least one of the first or second sheets comprising a flexible, air permeable fabric; b) a plurality of discrete sealed compartments formed by joining opposing interior surfaces of said containment structure such that each compartment is completely circumscribed by a continuous seal; c) a solid, particulate oxygen-activated exothermic composition located within the sealed compartments; and d) a removable environmental barrier structure. The continuous circumscribing seal surrounding each of the compartments further comprises a separation structure that permits partial or complete separation or detachment of a compartment from an adjacent compartment. The invention is particularly useful in medical procedures.

### FIELD OF THE INVENTION

The invention relates to the field of medical devices. In particular, the invention pertains to thermal devices for maintaining or controlling patient temperature.

### BACKGROUND OF THE INVENTION

A variety of thermochemical packs or pads are well known. Thermal packs can contain thermochemical compositions that, upon activation, generate cooling or warming temperatures depending upon the composition used. These packs can be applied to the surface, or attached to, the area of the body where the thermal therapy or effect is needed or desired. Thermochemical liquid packs can typically contain a liquid component physically separated from a solid component and designed to be combined at time of use by rupturing the physical barrier to initiate the admixture. One disadvantage of the liquid-solid thermochemical packs is that the larger the pack, the heavier their weight, and the longer the time period for thorough or complete activation to occur. Another disadvantage associated with liquid-type thermochemical packs is their duration of effect. For instance, most liquid packs afford thermal effect for about 30 minutes or less.

The importance of regulating, maintaining or controlling a patient's body temperature during and following medical procedures to avoid hypothermic body temperatures has been recognized as important. For example, when a patient is under anaesthesia, the body's ability to maintain body temperature can be compromised. Certain other benefits in regulating body temperature during surgical procedures have been recognized as well. It has been shown that maintaining body temperature enhances patient comfort, reduces the likelihood of surgical site infection, decreases hospital stay time, increases wound healing, and decreases blood loss.

As a result, patient warming systems have been developed to maintain body temperature during surgical procedures. The first systems developed to warm patients consisted of circulating water mattresses, which are still widely used today. One example of such a system is described in Fick U.S. Pat. No. 6,606,754. Since the patient lies on top of the circulating water mattress, the focus of heat transfer is to the posterior surface of the body. The anterior surface of the patient's body, on the other hand, is the surface which is most susceptible to heat loss. Another disadvantage of this system is the increased risk of burns to the patient's body as a result of the increased pressure of then patient's body surface at the contact points with the mattress. As far as placement of these mattresses on top of the patient, the water weight renders this option undesirable.

Various warm air convection blankets and systems have also been developed for surgical and rehabilitative applications. Examples of warm air convection blankets include those described in Kappel U.S. Pat. No. 6,241,756; Kappel et al. U.S. Pat. No. 6,156,058; Kappel U.S. Pat. No. 5,675,848; and Dickerhoffet al. U.S. Pat. No. 5,343,579, which describe inflated blankets wherein warmed air forced into an inflatable blanket wherein air exits onto the patient's body surface to warm. One disadvantage of these convection air blankets is that they are pre-configured for anatomy and particular procedure types and, therefore, are limited in their flexibility of use. Another disadvantage of such systems is that they require the use of secondary or additional non-disposable equipment, such as heaters and blower devices and tubing. Such secondary equipment can be noisy and cumbersome, and renders the successful operation of the blanket co-dependent upon the operation and working condition of the secondary devices. Furthermore, because of their design, relationships of interconnecting air channels or conduits must be preserved for their operation, which limits the ability to re-size at time of use as well as increase the likelihood of non-uniform temperature effects at different regions of the blanket. Warmed air blankets placed over the patient's body often require the use of additional securing devices, such as straps, to inhibit migration or movement of the blanket from the desired positioning.

One of the major disadvantages associated with prior art systems such as thermochemical packs, water current and convection air blankets is that practitioners must be mindful of punctures or other damage to the blanket containment caused by surgical tools and the like. Punctures and leakage of these systems can not only adversely affect the performance of the device, but introduce the internal contents of the device, e.g., thermochemical composition, into a sterile environment and compromise sterility.

Flexible thermal wraps containing iron compositions that activate upon exposure to air are also known. Thermal wraps of this type include a variety of designs, shapes and can contain multiple thermal cells. Examples of such thermal wraps are described in McNew U.S. Des. 344,343; Knight et al. U.S. Des. 467,006; Davis et al. U.S. Des. 407,822; Des. 412,751; Des. 433,145. One drawback of thermal wraps is that they are pre-configured for particular anatomical areas, such as the back or knee and therefore, have limited flexibility of use. Further, they include secondary attachment or positioning structures to secure them onto the user's body.

There exists a need in the field of thermal patient temperature control for improved thermochemical warming devices that are disposable and accommodate patient-specific and/or procedure-specific requirements. There is further a need for universally configurable thermal control devices that obviate the need to pre-order a size for a patient, as well as devices that are easily transportable with the patient for pre-operative patient warming. There is even further a need for patient warming devices suitable for use in medical procedures without requiring the accompaniment of secondary equipment requiring electricity.

### SUMMARY OF THE INVENTION

The invention provides a disposable, adjustable (or configurable) patient warming blanket comprising: a) a first blanket sheet and a second blanket sheet, said sheets being joined to one another around the perimeter to form a generally planar containment structure having a first side and second side and having an exterior surface and interior surface, at least one of said first or second sheets comprising a flexible, air permeable fabric; b) a plurality of discrete sealed compartments formed by joining opposing interior surfaces of said containment structure such that each compartment is completely circumscribed by a continuous seal; c) a solid, particulate oxygen-activated exothermic composition located within said sealed compartments; and d) a removable environmental barrier structure; wherein said continuous circumscribing seal surrounding each of said compartments further comprises a separation structure to permit partial or complete detachment of one or more of said compartments from one or more adjacent compartments.

The invention further provides a method of controlling patient body temperature in association with a medical procedure comprising the steps of: i) selecting a disposable, adjustable or configurable patient warming blanket of the above structure; ii) determining the extent of anatomical thermal coverage associated with the medical procedure; iii) removing the environmental barrier structure from the blanket thereby activating the exothermic composition; iv) adjusting the size and configuration of the blanket by separating at least one compartment from at least one adjacent compartment of the blanket; and v) positioning the blanket over the patient's body. Alternatively, the adjustment to the blanket can be made after positioning the blanket over the body. The method of the invention can be performed at one or more of pre-operative, operative, and post-operative time intervals.

According to the invention, it has been discovered that a thermal blanket can be constructed to be used in a surgical environment to control or maintain the patient's body temperature pre-operatively, during treatment and/or post-operatively, while at the same time be entirely composed of disposable materials. Furthermore, such a blanket can be structured to be presented as a single, universal device that can be readily adjusted, e.g., re-sized or re-configured, by the user to coordinate with a patient's specific anatomy, access requirement of a specific medical procedure, or both.

The blanket of the invention is not only devoid of several disadvantages associated with prior art devices, but offers many additional advantages and benefits, and combinations of advantages, that have been heretofore missing from such prior devices. Some of these further advantages are described as follows.

The blanket of the invention is easily transportable and can accompany the patient, and removes the need for secondary equipment, such as battery pack or electrically powered equipment. Consequently, the blanket of the invention can be used in circumstances where electricity is unavailable or inconvenient but emergency medical treatment is needed, such as ambulances, emergency helicopters, fire trucks or other emergency transportation means. Although medical or surgical environments is the preferred usage context of the invention where its advantages are most realized, the blanket of the invention can additionally be used in non-emergency circumstances, such as sporting events, camping, and the like.

Since the invention is in the form of a blanket and not a mattress, surface contact pressure points are reduced. As a result, the likelihood of burns is reduced as compared to circulating water or air mattress devices where the patient is placed on top of the mattress. Also as a result of being constructed as a blanket, thermal effect can be delivered to the anterior surface of the patient's body, which is the surface that experiences greater heat loss compared to the posterior surface.

Another advantage of the invention is the ease of operation and flexibility of its use. Because solid particulate, granular, or powdered air/oxygen-activated thermochemical compositions are employed in the invention, a plurality of discrete compartmentalized chambers containing these compositions can be activated uniformly and simultaneously by single step unpacking and exposing to air. This is not possible with thermochemical liquid compositions, wherein each compartment containing a liquid separated from a solid need to be individually and physically ruptured to combine and activate, or otherwise individually activated by manual trigger. This is further in contrast to a channeled conduit blanket using a single activation step, wherein the exothermic effect must await for the chemical reaction to “migrate” throughout the blanket, or facilitated by “shaking” or squeezing the blanket, which can take considerable extra time and effort to complete the activation of the full blanket.

Furthermore, the blanket of the invention can be designed for highly selective regional or isolated exposure to thermal effect. In addition to its separable compartmentalized structure, the blanket of the invention can be further constructed to permit partial activation of particular regions or groups of compartments, leaving remaining compartments unactivated.

### BRIEF DESCRIPTION OF THE DRAWINGS

The invention is further illustrated by the following drawings, none of which are to be construed as necessarily limiting the invention.

FIG. 1 is a top view of a blanket according to one embodiment of the invention.

FIG. 2 is a section of one corner of a blanket showing discrete compartments and a separation structure between according to one embodiment of the invention.

FIG. 3 is a top view of a blanket showing portions of the blanket separated from the remainder of the blanket according to one embodiment of the invention.

FIG. 4 is an angled side cross-sectional view of a portion of the blanket showing the interior of the compartments according to one embodiment of the invention.

FIG. 5 is a side cross-sectional view of an individual compartment showing the exothermic composition therein and layers of blanket material according to one embodiment of the invention.

FIG. 6 is a schematic view of the blanket placed over a human body and having portions removed for a medical procedure according to one embodiment of the invention.

FIG. 7 is an angled side view of the blanket of the invention shown folded and packaged within an environmental barrier structure according to one embodiment of the invention.

### DETAILED DESCRIPTION OF THE INVENTION

As used herein, the terms “adjustable” and “configurable” are meant to refer to the ability to adjust size, shape and/or appearance of the blanket by partial or complete detachment of compartments of the blanket that differ from its starting or initial dimensions and/or appearance. For example, the term is discussed in the context of the adjustability of the blanket in coordination with the patient's anatomy and/or surgical access requirements. The terms are intended to be distinguished from mere positioning of the entire device relative to a body.

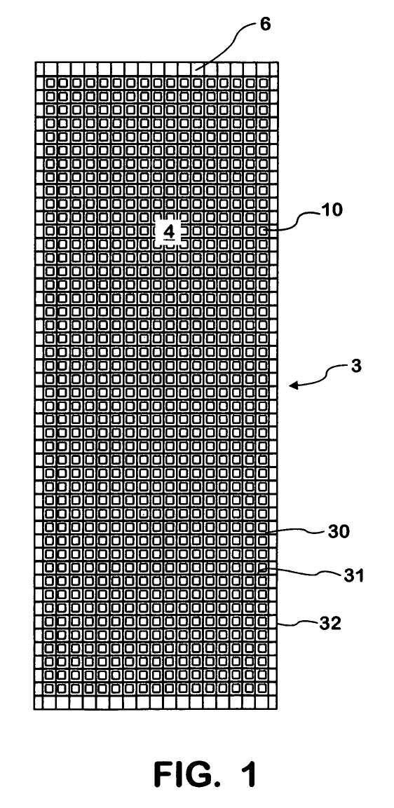

The overall configuration and dimensions of the blanket of the invention can vary, but it is preferred that the initially presented configuration, e.g., shape, and dimensions, e.g., length and width, be sufficient to cover at least one side of an adult human body. In other words, the overall length and width should permit full anatomical coverage of an adult human in reclined or supine position. Suitable starting dimensions and configurations for the blanket include, but are not limited to, a rectangular shape (as shown in FIG. 1, for example) or ovoid shape having a approximate length and width corresponding to that of a single side of an adult human body. Thus, a maximum body surface coverage possibility is afforded to the user as the blanket in its initially presented. Accordingly, with the full body as the starting configuration, a full range of re-sizing or adjustment options to the user are available. The blanket can be reduced in both size and location as needed within the range of a full body down to a single compartment. In other words, the blanket of the invention can be manufactured to be universally sized—one size fits all bodies and needs. From the initially presented starting blanket, the blanket can be both re-sizable and re-configurable. Since the appropriate adjustment is within the control of the user, the invention avoids the need to pre-order particular sizes of blankets for adults or infants, and further avoids the need to keep a multitude of devices in varying size ranges in inventory or storage.

The invention can have veterinary applications as well. Accordingly, the blanket of the invention can be manufactured and dimensioned according to the full body dimensions of a mammal, such as a horse, dog, cat, and the like.

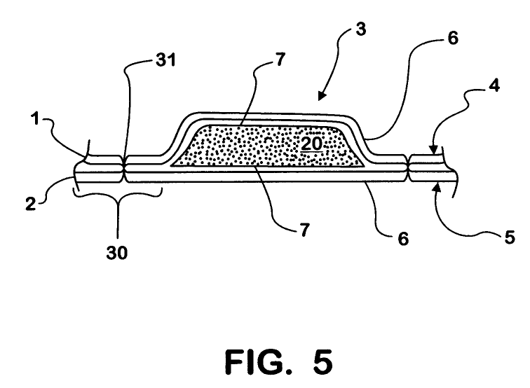

The blanket of the invention can be entirely constructed of disposable materials, both with respect to the blanket containment and the thermal composition within. Referring now to the figures—in particular FIG. 5, the blanket comprises a first sheet 1 and second sheet 2 joined to one another to form the blanket containment 3 having a first side 4 and second side 5 and exterior surface 6 and interior surface 7. In an alternative embodiment to two separate sheets being joined together about their perimeters, the blanket containment can be formed from a single folded sheet wherein the first side and second side are joined at the non-folded edges, so as to form a multilayer blanket structure with space between. For purposes of discussing the invention, the structure referenced as first sheet and second sheet is intended to include both separate sheet and single folded sheet embodiments.

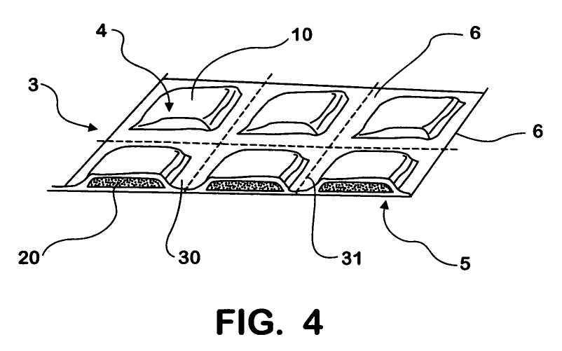

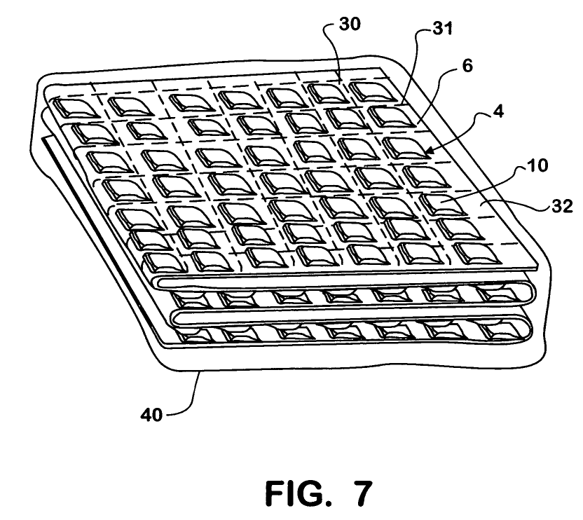

Each sheet of the blanket can be formed from a single layer of fabric, or multiple layers of fabric and/or film as illustrated in FIG. 5. At least one of the first or second sides 4 and 5 respectively is constructed to include an air permeable material. Thus, when the environmental barrier structure 40 (see FIG. 7) is removed or withdrawn from the blanket, air and oxygen is permitted to permeate across or through the fabric into the interior portion of a compartment 10 to interact with and activate the thermal composition 20 therein (see FIGS. 4 and 5).

The first and second sheet(s) 1 and 2 respectively, that form the blanket containment 3 can be formed by unwinding, positioning, and sealing two or more layers of nonwoven or film material so that there are distinct surfaces. A single sheet and its layers itself, or each respective sheet and its layers, can be composed of the same or different materials and/or arrangement of layers. Overall, the blanket containment 3 invention can be constructed from a uniform material and properties, or two different materials and/or different properties. One side of the blanket alone can be air permeable. Alternatively, both first and second sides 4 and 5 of the blanket can be constructed to be air permeable.

Suitable blanket materials for the invention include single or mutlilayer woven and non-woven fabrics and films. Non-woven fabrics can be formed from spunbond, meltblown, or combinations of both techniques (such as SMS), and can be composed of materials including polyethylene, polyester, nylon, polypropylene, and other polymeric materials.

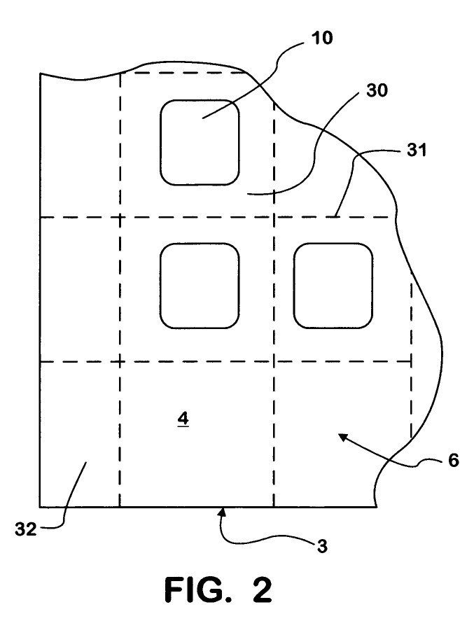

Referring now to FIGS. 2 and 4, seal(s) 30 can be formed using techniques and equipment readily available in the art, such as thermal or ultrasonic sealing techniques. The compartmental pattern of the blanket, such as the square-grid pattern illustrated in FIG. 1, can be formed from through the sealing process. The separation structure 31 can be coordinated with the sealing pattern. Suitable separation structures include attenuated or weakened regions within the seal pattern, represented in the figures as a dotted line of numerical reference 31. The location of the separation structure(s) can be varied, and can be produced throughout the entire sealing pattern of the blanket or, alternatively, single or multiple specific regions thereon. The separation structure 31 can be produced by perforation, scoring, thinning, and the like, along the length of the seal. Although the separation structure can be prepared by various techniques, it is important that when separation is affected by the user, a distinct, clean and controlled separation or parting occurs entirely within parameters of the seal per se or sealed region. The tear or separation should never encroach into the compartments.

Whether single layer or multilayer fabrics and materials are used, it is important to the invention that at least one side of the blanket comprise air permeable fabric composed of material or materials that permit air transport across or through the fabric into the compartment interior to activate the composition therein. Air transport across the fabric can be controlled or regulated by the particular materials used, and/or material thickness.

Referring now to FIG. 7, the blanket of the invention is packaged and contained within a removable environmental barrier structure 40 composed of a material which is impermeable to air and oxygen. Suitable environmental barrier structures can be constructed from materials including, but not limited to, single layer or multilayered plastics, metallic foils, coated films such as films coated with aluminum or silicon, and the like. The blanket can be sealed and packaged within the environmental barrier structure using conventional equipment and techniques readily available to those in the packaging field.

The compartments 10 formed by the sealing pattern can vary in size, shape and arrangement. The compartments 10 can be uniformly sized and/or shaped throughout the entire blanket, or two or more different compartment shapes and sizes can be used within a single blanket unit as well. As each particular compartment can differ in shape and dimension (length and width) from adjacent or surrounding compartments, wide variations are possible. Suitable compartment shapes include, but are not limited to, squares (as illustrated in the figures), rectangles, circles, ovals, triangles, trapezoids, symbols, logos, letters, numbers, and the like.

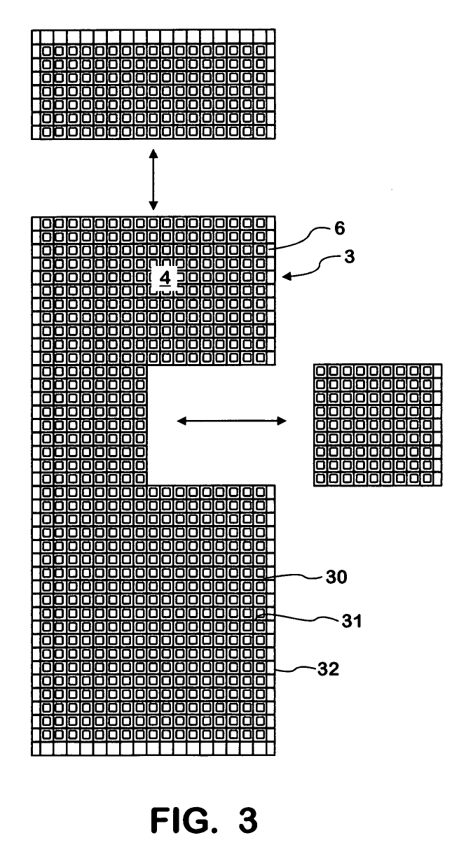

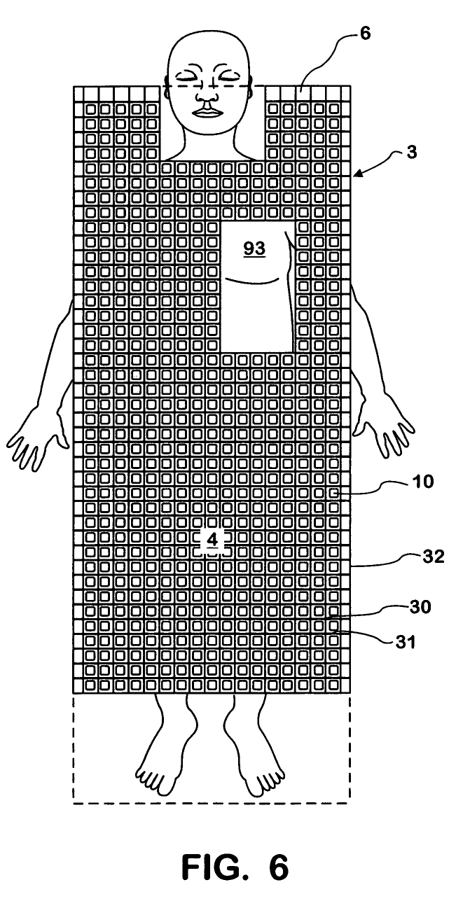

The location(s) of compartments 10 relative to the overall blanket structure can vary as well. The length and width of seams can also vary. The compartments can be positioned inward from the outer seal 32 and edges of the blanket as shown in FIGS. 1, 3 and 6. Compartments 10 and seals 30 (and outer seal) 32 can be configured to correspond to regional or partial separation and removal of blanket portions. As shown in FIG. 6, groups of compartments 10 and seals 30 can be structured to permit the creation of an inner window 33 completely surrounded by intact blanket. Compartments or groupings of compartments 10 can also be removed from remainder of blanket completely as shown in FIG. 3, or partially to form a flap still connected on one or more sides to the remainder of the blanket (not shown).

According to the invention, thermochemical compositions for use in the blanket of the invention are solid particulate air- or oxygen-activated exothermic compositions contained within the interior of each compartment 10. Upon exposure to air and oxygen, the exothermic reaction of the composition is initiated. A variety of oxygen-activated exothermic compositions suitable for use with the invention are readily available to those skilled in the thermal device field. Preferred for use with the invention are solid particulate oxygen-activated exothermic compositions comprising iron. Examples of suitable iron-containing exothermic compositions include, but are not limited to, those described in Yamashita et al. U.S. Pat. No. 3,976,049; Yamaguchi et al. U.S. Pat. No. 4,255,157; Abe U.S. Pat. No. 4,366,804; Usui U.S. Pat. No. 5,046,479; Ueki U.S. Pat. Nos. 5,342,412 and 5,366,492; and Burkett et al. U.S. Pat. No. 5,918,590, the entire texts of which are incorporated herein by reference.

Iron powder exothermic compositions are preferred due to their advantage of affording the blanket of the invention a lighter weight. The air-activated exothermic compositions can be formulated themselves, and/or the blanket containment can be designed, to control the temperature and/or duration of exothermic effect. Air-activated, solid, particulate iron containing exothermic compositions that can be used with the invention can comprise particulate or powdered iron as a primary ingredient in combination with secondary ingredients. Secondary ingredients that can be used include silicon, magnesium, carbon, sodium, aluminum, potassium and calcium, and mixtures thereof.

One example of a suitable composition for use in the blanket of the invention is that described in Usui U.S. Pat. No. 5,046,479. The air-activatable composition can comprise iron powder in an amount ranging from about 55% to about 65% of the total composition weight; water present in an amount ranging from about 18% to about 22% of the total composition weight, water retainer (such as charcoal or vermiculite) in an amount ranging from about 9% to about 11% of the total composition weight, activated carbon present in an amount ranging from about 3.5% to about 4.5% of the total composition weight, and salt present in an amount ranging from about 4.5% to about 6% of the total composition weight.

An important aspect of the invention is that the user is afforded a variety of adjustment and activation options based on the construction of the blanket, only a few of which are illustrated in FIGS. 3 and 6. In another embodiment (not shown), for example, the blanket of the invention can be constructed to include a plurality of removable environmental barriers within a single blanket unit. Thus, either complete or partial activation is permitted by regionally selective exposure to air and activation, leaving the remainder inactivated portions of the blanket intact. Further associated with this embodiment is the capability to sequentially activate various regions of the same blanket, or anatomically relocating thermal effect on the user's body within a single usage session.

The blanket of the invention can also be manufactured in anticipation of specific surgical procedures in other ways as well. For example, the fabric used to construct the blanket containment can be color-coded to afford the user a readily identifiable visual indication of its intended use. In another embodiment, the compartments of the blanket can be shaped in alignment or in cooperation with particular surgical access requirements associated with a specific surgical procedure.

In yet a further embodiment, supplemental structures can be added in contemplation of specific applications or procedures. Examples of supplemental structures that can be used in conjunction with the blanket include, but are not limited to, ample, attachable drapes, straps, fasteners, tapes, and the like. Supplemental structures can be pre-attached to the blanket and/or attached to the blanket at time of use.

Although the preferred embodiment is a universal starting configuration, the blankets of the invention can also be designed for use in conjunction with specific procedures. An alternative and less preferred embodiment is for the initial size of the blanket to be dimensioned according to half of the body, e.g., upper body (waist to shoulders) or lower body (waist to ankles/feet) length and width. This embodiment is an option, but compromises the “one-size-fits-all” advantage relative to a starting configuration having full human body dimensions.

The blanket of the invention can be prepared using conventional equipment and techniques readily available to those skilled in the medical fabric field. One of the important advantages of the invention the ease of manufacture capability. The ease of manufacture is, of course, directly related to the actual product design to be employed.

As the blanket of the invention can be configured to accommodate the patient's anatomy, the extent of body coverage desired, or body access requirements of a particular surgical procedure, the modification or adjustment of the blanket can be contemplated in advance of its use. The activation of the blanket, i.e., the exposure of the blanket to air and oxygen, should be reserved until near or at the time of its actual use.

The invention includes a method of controlling patient body temperature in association with a medical procedure. In other words, the blanket of the invention can be used at one or more of the pre-operative, operative, and/or post-operative time intervals to effect therapy.

In one example, as the patient is prepared for a medical procedure or surgery, the patient warming blanket of the invention is removed from its packaging and environmental barrier structure. Upon partial or complete removal of the environmental barrier structure surrounding the blanket, environmental air and oxygen begin to transport across the air permeable material and into the exposed compartments containing the air-activated exothermic composition therein. The exothermic reaction is initiated and the thermal warming effect begins.

The extent of anatomical coverage suitable for the medical procedure can then be determined. The blanket can then be adjusted, e.g., sized and/or configured, according to the preferences or requirements of the particular surgical procedure to be carried out, as well as according to the patient's particular anatomy. The adjusting of the blanket by separation and/or removal of regions of the blanket can take place before positioning the blanket over the body, or alternatively, after the blanket has been positioned over the body.

According to the invention, the removal of portions or the blanket can be initiated at the edge of the blanket at one or more locations as shown in FIG. 3, or alternatively, at the central region of the blanket, e.g., to form a window or fenestration surrounded by the remaining intact blanket as shown in FIG. 6. To remove a portion of the blanket, the user can cut or separate along the separation structure of the seam until the portion to be removed is physically separated from the remaining intact blanket.

Alternatively, a portion of the blanket can be partially separated and folded over to avoid obstruction during the procedure, for example. Thus, the invention also provides the advantage providing a single adjustable blanket that, despite its adjustability, can be maintained as a single contiguous unit without necessitating the generation of “scraps” in the surgical environment. Upon completion of the procedure or otherwise when the blanket is no longer necessary, the blanket can be disposed.

### INDUSTRIAL APPLICABILITY

The invention is useful in the medical field whenever controlling or maintaining body temperature is important to the success or optimal results of a medical procedure. The blanket of the invention is particularly useful in invasive surgical procedures where the patient is anaesthetized and body temperature is expected to drop, wherein the application of the blanket can impart the counter-thermal exothermic warming effect to prevent hypothermia.

The invention has been described herein above with reference to various and specific embodiments and techniques. It will be understood that reasonable variations and modifications can be made of such embodiments and techniques without significant departure from either the spirit or scope of the invention defined by the claims below.

What is claimed is:

1. A disposable, configurable patient warming blanket comprising:
a) a first sheet and a second sheet, said sheets being joined together about the perimeter to form a generally planar containment structure having a first side and second side and an exterior surface and interior surface, at least one of said sheets comprising a flexible, air permeable fabric;
b) a plurality of discrete sealed compartments formed by joining opposing interior surfaces of said containment structure such that each compartment is completely circumscribed by a seal;
c) a solid, particulate oxygen-activated exothermic composition located within each of said sealed compartments; and
d) a removable environmental barrier structure;
wherein said seal circumscribing said compartments comprises a separation structure to permit partial or complete detachment of one or more of said compartments from one or more adjacent compartments.

2. The blanket according to claim 1, wherein said blanket is dimensioned to cover the full body of a human adult.

3. The blanket according to claim 1, wherein said blanket is dimensioned to cover the full body of a mammal.

4. The blanket according to claim 1, wherein one side of said first and second sides is composed of air-permeable material.

5. The blanket according to claim 1, wherein both of said first and second sides are composed of air permeable material.

6. The blanket according to claim 1, wherein said blanket containment is constructed from two separate sheets.

7. The blanket according to claim 1, wherein said blanket containment is constructed from a single folded sheet.

8. The blanket according to claim 6, wherein one of said sheets is composed of a material different than the other sheet.

9. The blanket according to claim 1, wherein at least one of said sheets is composed of a single layer fabric.

10. The blanket according to claim 1, wherein at least one of said sheets is composed of a multilayer fabric.

11. The blanket according to claim 1, wherein said solid, particulate oxygen-activated exothermic composition comprises iron powder.

12. A method of controlling patient body temperature in association with a medical procedure comprising the steps of:
i) selecting a disposable, configurable patient warming blanket comprising:
a) a first sheet and a second sheet, said sheets being joined together about the perimeter to form a generally planar containment structure having a first side and second side and an exterior surface and interior surface, at least one of said sheets comprising a flexible, air permeable fabric;
b) a plurality of discrete sealed compartments formed by joining opposing interior surfaces of said containment structure such that each compartment is completely circumscribed by a seal;
c) a solid, particulate oxygen-activated exothermic composition located within each of said sealed compartments; and
d) a removable environmental barrier structure;
wherein said seal circumscribing said compartments comprises a separation structure to permit partial or complete detachment of one or more of said compartments from one or more adjacent compartments;
ii) determining the extent of anatomical thermal coverage associated with said medical procedure;
iii) removing said environmental barrier structure from said blanket thereby activating said exothermic composition;
iv) adjusting the size and configuration of said blanket by separating at least one compartment from at least one adjacent compartment of said blanket; and;
v) positioning said blanket over the patient's body.

13. The method according to claim 12, wherein said method is performed pre-operatively.

14. The method according to claim 12, wherein said method is performed during said medical procedure.

15. The method according to claim 12, wherein said method is performed post-operatively.

16. A method of controlling patient body temperature in association with a medical procedure comprising the steps of:
i) selecting a disposable, configurable patient warming blanket comprising:
a) a first sheet and a second sheet, said sheets being joined together about the perimeter to form a generally planar containment structure having a first side and second side and an exterior surface and interior surface, at least one of said sheets comprising a flexible, air permeable fabric;
b) a plurality of discrete sealed compartments formed by joining opposing interior surfaces of said containment structure such that each compartment is completely circumscribed by a seal;
c) a solid, particulate oxygen-activated exothermic composition located within each of said sealed compartments; and
d) a removable environmental barrier structure;
wherein said seal circumscribing said compartments comprises a separation structure to permit partial or complete detachment of one or more of said compartments from one or more adjacent compartments;
ii) determining the extent of anatomical thermal coverage associated with said medical procedure;
iii) removing said environmental barrier structure from said blanket thereby activating said exothermic composition;
iv) positioning said blanket over the patient's body; and
v) adjusting said blanket by separating at least one compartment from at least one adjacent compartment of said blanket.

17. The method according to claim 16, wherein said method is performed pre-operatively.

18. The method according to claim 16, wherein said method is performed during said medical procedure.

19. The method according to claim 16, wherein said method if performed post-operatively.
